(19) United States
(12) Patent Application Publication
(10) Pub. No.: US 2008/0068932 A1
(43) Pub. Date: Mar. 20, 2008

(54) Wrist watch for monitoring diabetes

(76) Inventor: Bennie Mosley, Rancho Cucamonga, CA (US)

Correspondence Address: Law Office of J. Curtis Edmondson, 15490 NW Oak Hills Drive, Beaverton, OR, 97006, US

(21) Appl. No.: 11/897,850

(22) Filed: Sep. 1, 2007

Related U.S. Application Data
(60) Provisional application No. 60/844,282, filed on Sep. 14, 2006.

(51) Int. Cl. A44C 5/00; A61B 5/00; G04B 47/00; G04B 47/06

(52) U.S. Cl. 368/281; 368/11; 368/12; 600/301; 600/316

(57) ABSTRACT

A diabetes monitoring watch is shown with a computing system located in the watch, including a glucose monitoring system, a pulse rate monitor, a body temperature monitor, and an alert system. The system looks at the glucose level, the body temperature, and the heart rate and determines if the diabetic is in need of assistance. The diabetes monitoring watch also has an alert system for notification of the individual or other individuals if the person is in need.

### CROSS REFERENCE TO RELATED APPLICATIONS

This application claims the benefit of U.S. Provisional Patent Application No. 60/844282 filed Sep. 14, 2006.

### BACKGROUND OF THE INVENTION

1. Field of the Invention

The present invention relates to a wrist watch for monitoring the overall health of a diabetic patient.

2. Discussion of the Related Art

Diabetes affects millions of Americans and tens of millions world-wide. Diabetes is the inability of the body to properly metabolized glucose resulting in hyperglycemia. The long term physical manifestations of hyperglycemia are pronounced: including, but not limited to heart disease, atherosclosis, blindness, stroke, hypertension and kidney failure.

Hyperglycemia is present when the blood glucose levels consistently exceed 126 mg/dL or 7 mmol/L. (see http://en.wikipedia.org/wiki/Hyperglycemia#Measurement and definition). When hyperglycemia becomes severe, patients may develop one of two potentially life-threatening complications: diabetic ketoacidosis (DKA) or hyperglycemic hyperosmolar nonketotic syndrome (HHNS). (See http://findarticles.com/p/articles/mi_ga3689/is_{—}200108/ai_n8967972).

The increase in the risk of heart disease for chronic suffers of hyperglycemia can lead to an increase in the risk of heart attacks. A symptom of the onset of a myocardial infarction (e.g. heart attack) being an increased or irregular heart rate. Also during exercise by a diabetic the monitoring of heart rate can be used to roughly calculate the amount of carbohydrates to ingest to maintain proper blood sugar levels. (see http://www.idea2000.org/moreinfo/docs/diabetes_polar_tips.html).

One method of maintaining blood glucose levels involves determining the caloric count of foods and then determining the amount of food to eat to maintain the proper blood sugar level ( see http://www.endocrineweb.com/diabetes/treatment.html).

The prior art describes systems for predicting blood glucose levels. U.S. Pat. No. 6,188,648 (Feb. 13, 2001) to Olsen describes a wristwatch device where the person can manually calculate carbohydrate counts. U.S. Pat. No. 7,215,601 B2 (May 7, 2007) to Plancon, et. al depicts a wristwatch display system that conveys analog information on the face of a wristwatch.

The prior art also describes systems for non-invasive monitoring glucose levels that may be incorporated into a wristwatch. U.S. Pat. No. 7,174,199 B2 (Feb. 6, 2007) to Berner, et. al describes a method of monitoring glucose levels specifically in the blood. U.S. Pat. No. 7,084,976 (Aug. 1, 2006) to Morokawa, et. al depicts an earring like device that monitors glucose concentration levels. U.S. Pat. No. 7,147,153 B2 (Dec. 12, 2006) to Rowe depicts a multispectral glucose monitoring system.

The prior art describes monitoring the caloric expenditure rate of the individual. U.S. Pat. No. 6,571,200 (May 27, 2003) to Mault describes a computer based apparatus that determines caloric expenditure based on activity monitors and a GPS input. U.S. Pat. No. 7,220,220 (May 22, 2007) to Stubbs, et. al depicts an exercise monitoring system which monitors physiological signals and incorporates a GPS unit.

The prior art also describes predictive algorithms for determining the onset of hypoglycemia. See U.S. Pat. No. 6,923,783 B1 (Aug. 2, 2005) to Kovatchev, et. al describing a non-linear model and implementation for hypoglycemia.

None of the aforementioned devices provide a wearable diabetes monitoring system which can predict when person may need to take food at a particular time and can notify the person when their blood sugar exceeds a particular range.

### SUMMARY OF THE INVENTION

The present inventive subject matter overcomes problems in the prior art by providing a diabetic monitoring wristwatch with the following qualities, alone or in combination.

The present inventive concept monitors three physiological factors associated with being a diabetic and integrates this monitoring function into a single wristwatch.

The diabetes monitoring watch is attached to the wrist of an individual, and continuously monitors the individual's blood glucose levels. If a combination of the blood glucose levels of the individual exceed a preset band of acceptable values, in combination with other physiological parameters, such as the: rate of heartbeat, temperature, the watch will alert the individual or other parties of this condition. This notification can be done either by sound, vibration, or by activation of other devices through an electronic interface.

The wristwatch will also be water resistant, to allow use while involved in sporting activities or while showering.

The wristwatch will also be aesthetically pleasing, so as to not alert other people that the individual may suffer from any number of glucose related maladies.

These and other embodiments are described in more detail in the following detailed descriptions and the figures.

The foregoing is not intended to be an exhaustive list of embodiments and features of the present inventive subject matter. Persons skilled in the art are capable of appreciating other embodiments and features from the following detailed description in conjunction with the drawings.

### BRIEF DESCRIPTION OF THE DRAWINGS

FIG. 1 is a front view of the diabetic monitoring watch.

FIG. 2 is a rear view of the diabetic monitoring watch.

FIG. 3 is a systems diagram of the diabetic monitoring watch.

FIG. 4 is a software flow chart of the diabetic monitoring watch.

### DETAILED DESCRIPTION

While describing the invention and its embodiments various terms will be used for the sake of clarity. These terms are intended to not only include the recited embodiments, but also all equivalents that perform substantially the same function, in substantially the same manner to achieve the same result.

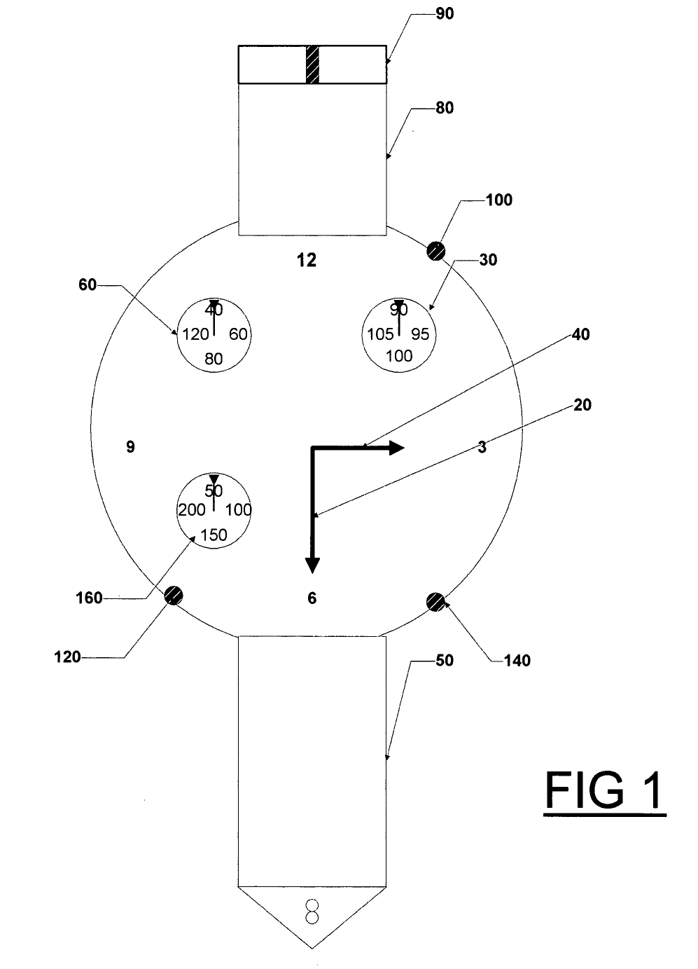

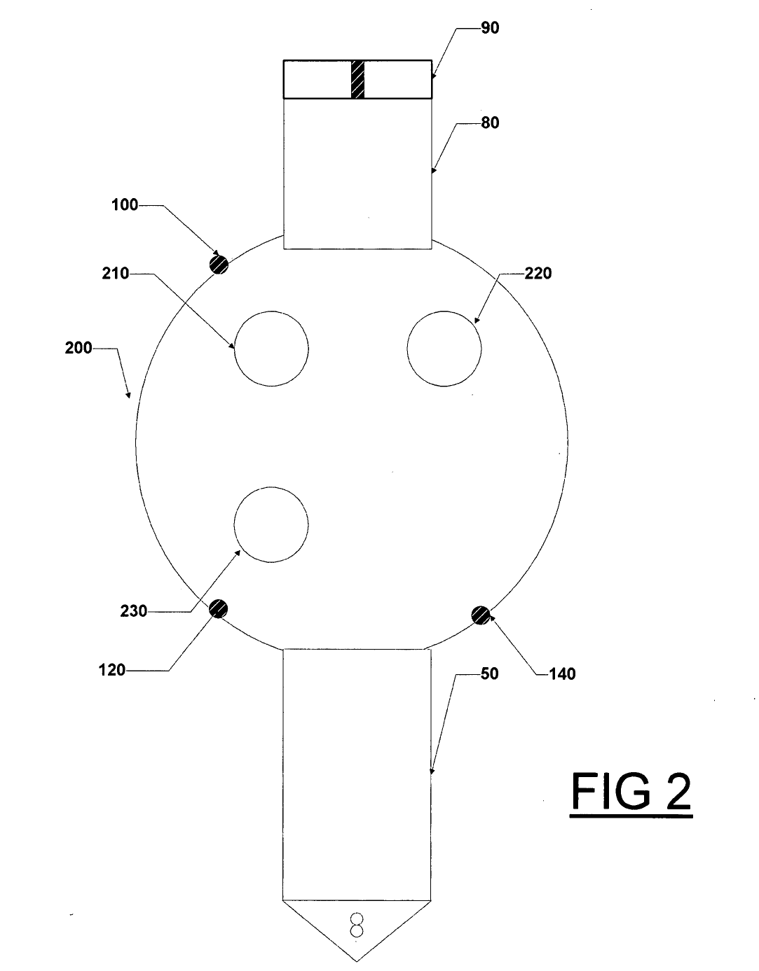

FIG. 1 illustrates the face of the watch 10. FIG. 2 illustrates the back of the watch 200. The face of the watch 10 consists of the following metabolic monitoring features: temperature display 30, pulse rate monitoring 60, and glucose monitor 160.

The body temperature display 30 on the watch 10 consists of a dial that ranges from a low body temperature to a high body temperature. In this embodiment the body temperature display 30 ranges from 90 to 105 degrees F. A more narrow range of body temperatures may be displayed in other embodiments, for example from 98 to 103 degrees F. In other embodiments a digital display for the body temperature may be used in lieu of a dial display. The body temperature is measured by the temperature sensor 210 mounted on the back of the watch 200.

The pulse rate monitor display 60 on the watch 10 consists of a dial that ranges from a low pulse rate to a high pulse rate. In this embodiment the pulse rate monitor ranges from 40 beats per minute to 120 beats per minute. A more narrow or wider range of pulse rates may be displayed in other embodiments. In other embodiments a digital display for the pulse rates may be used in lieu of a dial display. The pulse rate is measured by the pulse rate sensor 220 mounted on the back of the watch 200.

The glucose level indicator 160 on the watch 10 consists of a dial that ranges from a low amount to a high amount. In this embodiment, the glucose level ranges from 50 to 250 mg/dl. A more narrow or wider range of glucose level displays may be displayed in other embodiments. In other embodiments a digital display for the glucose levels may be used in lieu of a dial display. The glucose level is measured by the transdermal glucose sensor 230 mounted on the back of the watch 200.

The internal functions of the watch are configured through a twelve alarm button 100, and the medical data bank button 140.

The watch also consists of the ability to display time with a second hand 20, and an hour hand 40. Other embodiments include a digital display in lieu of a second and hour hand configuration.

The watch is affixed to the person with straps 50, 80. The strap 50 is inserted into strap 80 in a looping fashion and held by a buckle or a hook and pile assembly (such as Velcro).

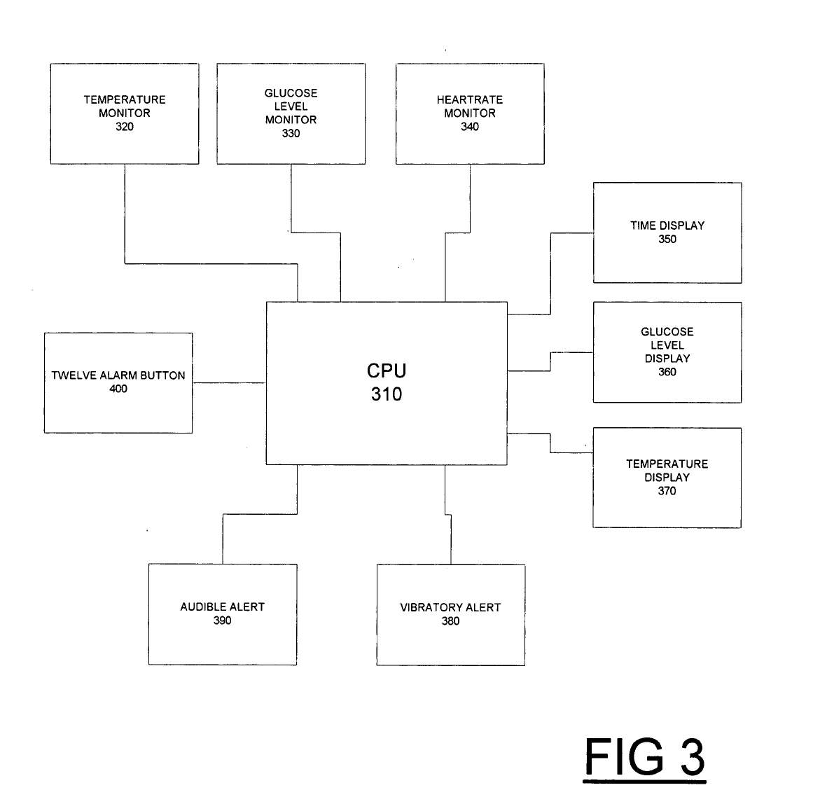

Now referring to FIG. 3 which shows a block diagram of the glucose monitoring wristwatch.

The CPU 310 is the central processing unit located within the wristwatch. A number of CPU's 310 that are well known in the arts may be used, including, but not limited to, 16 bit and 32 bit microprocessors. The CPU 310 also incorporates Read Only Memory (not shown) for stored program instructions and Random Access Memory (not shown) for operational memory. The CPU 310 may be connected to peripheral devices via a direct connection or using bus configuration.

The CPU 310 is connected to a temperature interface 320, a glucose level interface 330, and a heart rate monitor 340. The CPU is connected to a time display 350, a glucose level display 360, and a temperature display 370. Also connected is a vibratory alert 380 and a auditory alert 390. Controlling the system is the twelve alarm button 400.

The temperature interface 320 monitors body temperature using a direct contact with the skin. The temperature interface 320 is a dermal contact device located on the rear of the watch shown as temperature sensor 210 in FIG. 2. The temperature interface is of a type similar to the MiniMitter Vitalsense Skin Temperature Patch (see http://www.minimitter.com/ProductsNitalSense/temperature.html#patch). The temperature interface 320 converts the body temperature represented by an analog electrical signal into a digital value that can be processed by the CPU 310.

The glucose level interface 330 monitors glucose levels by a transdermal glucose sensor 230 mounted to the back of the watch 200 as shown in FIG. 2. The glucose level interface 330 transdermal glucose sensor 230 is of a type similar to the Glucowatch®.

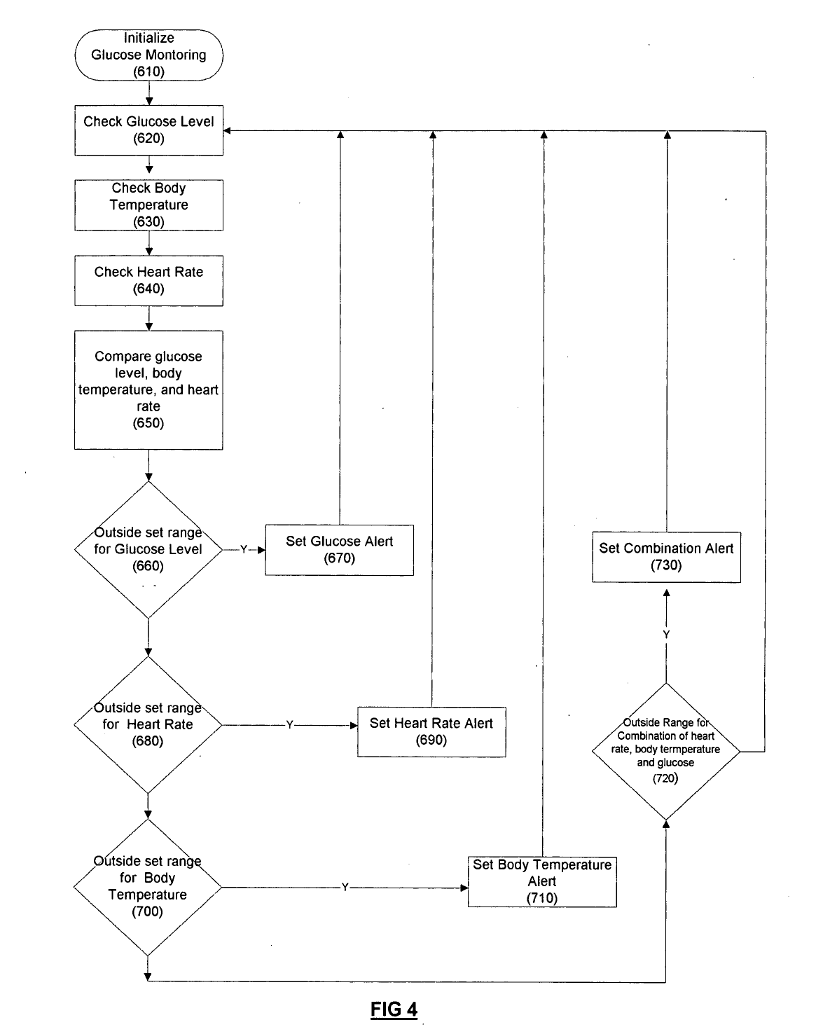

Now referring to FIG. 4 which shows a flowchart of the operation of the glucose-monitoring wristwatch. In the first step, the system is initialized 610. The system checks the glucose level 620. The system checks the body temperature 630. The system checks the heart rate 640. The system then compares the glucose level, body temperature, and heart rate 650 via an internal algorithm. If the glucose level is outside the set range 660 then the watch will send a glucose alert signal 670 and repeat the process. If the heart rate is outside the set range 680 then the watch will send a heart rate alert signal 690. If the body temperature is outside the set range 700, then the watch will send a body temperature alert signal. If a combination of the heart rate, body temperature, and glucose levels are outside the combined set ranges 710, then an alert is set.

The system is configured by using the twelve alarm button. The configuration using a single button is well known in the arts and consists of a series of button presses to set time, date, glucose ranges, heart rate ranges, and body temperature ranges. The twelve alarm button also permits the setting of interval alerts to notify the person to take medications, in particular medications that aide in the process of glucose monitoring.

I claim:

1. A diabetes monitoring watch comprising:
a) a time piece, wherein the time piece is configured to fit on ones wrist; and wherein said time piece has a face and a back;
b) a central processing unit, the central processing unit comprising a stored programmable memory, a random access memory, a programmable central processing unit; and
c) a glucose monitor, wherein the glucose monitor is coupled to the central processing unit;
d) a heartbeat monitor; wherein the heartbeat monitor is coupled to the central processing unit;
e) a body temperature monitor, wherein the body temperature monitor is coupled to the central processing unit;
f) a pulse rate monitor, wherein the pulse rate monitor is coupled to the central processing unit;
g) an alert system, where the alert system is coupled to the central processing unit;
and wherein the central processing unit is configured to read the blood glucose level from the glucose monitor and store a multiplicity of blood glucose levels in random access memory; and wherein the blood glucose level is displayed on the face of the time piece;
and wherein the central processing unit is configured to read the pulse rates from the pulse rate monitor and store a multiplicity of pulse rates in the random access memory; and wherein the pulse rate is displayed on the face of the time piece;
and wherein the central processing unit is configured to read the body temperature from the body temperature monitor and store a multiplicity of body temperatures; and wherein the body temperature is displayed on the face of the time piece;
and wherein the central processing system sets an alert level in the alert system as calculated from the blood glucose levels, pulse rates, and body temperatures as stored in the central processing unit.

2. A diabetes monitoring watch as in claim 1, wherein the glucose monitor reads the glucose levels using a transdermal sensor.

3. A diabetes monitoring watch as in claim 1, wherein the glucose monitor reads the glucose levels using an infrared sensor.

4. A diabetes monitoring watch as in claim 1, wherein the body temperature that is displayed on the timepiece is analog.

5. A diabetes monitoring watch as in claim 1, wherein the alert system sends a notice using vibration.

6. A diabetes monitoring watch as in claim 1, wherein the alert system sends a notice using electronic mail.

7. A diabetes monitoring watch as in claim 1, wherein the alert system sends a notice using an audible tone.

8. A diabetes monitoring watch as in claim 1, wherein the pulse rate display on the face of the time piece is analog.

9. A diabetes monitoring watch as in claim 1, wherein the body temperature display on the face of the time piece is analog.
